(19) United States
(12) Patent Application Publication
(10) Pub. No.: US 2010/0028894 A1
(43) Pub. Date: Feb. 4, 2010

(54) Photographic Determination of Analytes

(76) Inventors: Thomas Carell, Krailling (DE); Anja Schwögler, Mannheim (DE)

Correspondence Address: BRINKS, HOFER, GILSON & LIONE, P.O. BOX 1340, MORRISVILLE, NC, 27560, US

(21) Appl. No.: 12/514,097

(22) PCT Filed: Nov. 7, 2007
(86) PCT No.: PCT/EP07/09669

§ 371 Date: May 8, 2009

(30) Foreign Application Priority Data
Nov. 9, 2006 (EP) 06 023 369.9

(51) Int. Cl. C12Q 1/68; G01N 21/00; G01N 33/00

(52) U.S. Cl. 435/6; 436/164; 436/94

(57) ABSTRACT

The present invention refers to a detection method for analytes using the principle of black-and-white photography and to reagent kits for performing the method, furthermore applied this new technology to detect a biologically relevant sequence in the nanomolar range (femtomoles) in an application circumventing the necessity of a PCR. There are still numerous ways to optimize this methodology that is suitable for a large variety of applications in the genomic diagnostics and proteomics areas.

The present invention refers to a detection method for analytes using the principle of black-and-white photography and to reagent kits for performing the method. This new technology may be applied to detect a biologically relevant nucleic acid sequence in the nanomolar range in an application circumventing the necessity of a PCR. The methodology is suitable for a large variety of applications in the genomic diagnostics and proteomics areas.

### INTRODUCTION

There is a great need in the medical, scientific and non-scientific community for rapid and simple diagnostic assays able to detect biomaterials such as oligonucleotides, DNA, RNA and proteins. The methodologies available today require expensive equipment and technologies and they are exclusively suited for specialized users. In the case of DNA detection the polymerase chain reaction [1] (PCR) or comparable target-amplification methods are still the most widely used for their reliability and sensitivity (5-10 DNA molecules). In some cases these methods exhibit shortcomings in terms of specificity and require an expensive multi component assay. Direct detection methods were developed recently using complex technologies such as fluorescent, chemoluminescent, electrochemical, radioactive processes, or sophisticated materials such as nano-particles [2-8]. Although these new assays can detect selected oligonucleotides in the pico-, femto- and even atto-molar range, their application requires a specific scientific background thus limiting the method to highly specialized labs.

A novel approach to detect DNA and RNA without any specific scientific background would be a landmark result in order to extend these kinds of diagnostics to a large variety of applications. This proposed method should cover the fields of human in vitro diagnostics such as testing for infectious and Bioterrorism agents or genetic testing, oncology, research and many more. The aim of the present invention is to develop an easy to use method for all these fields without the involvement of sophisticated and expensive instrumentation.

The irradiation of a photopaper or of an emulsion containing silver halide crystals generates Ag₄ nuclei as latent images [9]. Those clusters are selectively enlarged by the subsequent reductive development process. This development step can be seen as the amplification of the original signal—the latent image—by a factor of 10¹¹. The sensitivity of such emulsions or papers is called “intrinsic sensitivity” and is limited to wavelengths absorbed by the silver halide. The process called spectral sensitization induces sensitivity to the longer wavelength of the visible spectrum using dyes called spectral sensitizers adsorbed to the emulsion grains [10]. Cyanine, merocyanine and pinacyanol dyes constitute the majority of spectral sensitizers employed thus far, though many other molecules were used in photography before the cyanines were recognized as the best class of dyes for this application [11].

Page et al. (PNAS, 1996, 14020-14024) describe the use of radioactive oligonucleotide probes to examine Alzheimer disease and control brain expression of a disease-associated mRNA. In Situ hybridization followed by gamma-ray irradiation generated by ³⁵S containing oligonucleotides forms an image on an X-ray film or nuclear emulsion (autoradiography). This technique involves the use of radioactive material and long contact-time of 15-30 days between the radioactive material and the X-ray film. The imprinting process involves the interaction of beta particles or gamma rays with the special X-ray film. It differs from the formation of a latent image in a photographic process which catalyzes the deposition of Ag on the photopaper (or a light sensitive medium).

U.S. Pat. No. 4,139,388 describes a medium containing a photosensitizer, but the document does not contain any suggestion for diagnostic applications. Further, the photosensitizer is not coupled to a reporter molecule.

PCT/EP2006/004017 discloses a method for highly sensitive DNA detection which is accessible in many fields even for non-specialized users, without the need for a professional lab and in a very simple way. According to this method, an oligonucleotide or a DNA double strand is labelled with a photosensitizer used in photography. A solution containing this labelled oligonucleotide (ODN) is spotted on photographic paper. Even without any spectral sensitization, the method allows a detection of the labelled DNA in a picomolar sensitivity (300 attomoles) after irradiation and development of the photopaper. The content of this document is herein incorporated by reference.

The present invention refers to the use of modified photographic media, e.g. photographic media without or with modified supercoats, with modified light-sensitive silver halide crystals or with a modified matrix.

The present invention relates to a method for detecting an analyte in a sample comprising the steps:
(i) providing a sample,
(ii) providing a reporter molecule comprising a photosensitizer group or a handle group for introducing a photosensitizer group,
(iii) contacting the sample with the reporter molecule,
(iv) if necessary, reacting the handle group with a reaction partner comprising a photosensitizer group,
(v) irradiating said reporter molecule in contact with a modified photosensitive medium under conditions wherein marker groups are formed in said modified photosensitive medium depending on the binding of the reporter molecule to the analyte, and
(vi) detecting said marker groups.

Further, the invention refers to a reagent kit for detecting an analyte in a sample comprising
a) a reporter molecule comprising a photosensitizer group or a handle group for introducing a photosensitizer group,
b) optionally a reaction partner for the handle group comprising a photosensitizer group and
c) a modified photosensitive medium which forms marker groups upon irradiation of unquenched photosensitizer groups.

The present invention allows a highly sensitive detection of analytes, e.g. nucleic acids or nucleic acid binding proteins, in biological samples, e.g. clinical samples, environmental samples or agricultural samples. Preferred applications include, but are not limited to, the detection of genetic variabilities, e.g. single nucleotide polymorphisms (SNPs), pesticide or medicament resistances, tolerances or intolerances, genotyping, e.g. the detection of species or strains of organisms, the detection of genetically modified organisms or strains, or the detection of pathogens or pests, and the diagnosis of diseases, e.g. genetic diseases, allergic diseases, autoimmune diseases or infectious diseases. A further preferred application is the detection of nucleic acids in samples for brand protection, wherein products such agricultural products, food products, or goods of value and/or packaging of these products are encoded with product-specific information, e.g. but not limited to production site, date production, distributor etc., and wherein this information is detected with the method as described above.

### DETAILED DESCRIPTION OF PREFERRED EMBODIMENTS

The present invention comprises the detection of an analyte. The detection may be a qualitative detection, e.g. the determination of the presence or absence of an analyte, e.g. a specific nucleic acid sequence in the sample to be analysed. The invention, however, also allows quantitative detection of an analyte, e.g. a nucleic acid sequence, in the sample to be analysed. Qualitative and/or quantitative detection may comprise the determination of labelling groups according to methods known in the art.

The analyte to be detected is preferably selected from nucleic acids and nucleoside-, nucleotide- or nucleic acid-binding molecules, e.g. nucleoside-, nucleotide- or nucleic acid-binding proteins. More preferably, the analyte is a nucleic acid, e.g. any type of nucleic acid which can be detected according to known techniques, particularly hybridization techniques. For example, nucleic acid analytes may be selected from DNA, e.g. double-stranded or single-stranded DNA, RNA, or DNA-RNA hybrids. Particular examples of nucleic acid analytes are genomic DNA, mRNA or products derived therefrom, e.g. cDNA.

The present invention refers to the detection of an analyte using a modified photosensitive medium. In the following, preferred embodiments of the invention are described.

Traditional photopaper is protected with a supercoat, which is typically gelatine. In order to detect an analyte, e.g. DNA, the molecule has to diffuse through this material in order to reach the light sensitive layer. In practice the supercoat is designed to keep dust and unwanted sensitizer away from the light sensitive surface. Optimizing photopaper for DNA analyte detection on light sensitive surfaces requires either to fully or partially eliminate the supercoat and/or to change it into a material which can be better penetrated by the analyte, e.g. DNA. For example, a material that actively binds DNA to transport it to the surface like DNA counterstrand containing materials or generally positively charged species either in the coat and/or a coat made out of positively charged molecules, e.g. polymer molecules, is possible. Further, an active transport of a charged analyte or analyte/reporter molecule complex, e.g. by applying an electrical field into the light-sensitive layer is feasible.

In order to achieve maximum sensitivity is desirable to optimize the size and the shape of the light sensitive silver halide crystals. In addition the crystals can be doted to become more sensitive. Crystal size determines how much Ag can be generated from one Ag cluster nucleus present in or on the crystal. In this way the crystal size determines the amplification factor. Similarly the density of the silver halide crystals will determine the sensitivity. With high density material, the development of one crystal might jump over to neighbouring crystals or one DNA molecules labelled with one or more chromophores can sensitize more crystals in its vicinity. The present invention refers to the use of modified silver halide, e.g. AgBr crystals having a spherical, a red-shaped or an irregular morphology or to mixtures thereof. Further, the invention relates to the use of surface-modified, e.g. sulphide-etched crystals.

The matrix which holds the silver halide crystals also requires optimization in a similar way to the supercoat. The more dense the material is the more difficult will it be particularly for long labelled DNA strands to reach the light sensitive crystals. A loose connection of the crystals using less crosslinked or non-crosslinked material, e.g. gelatine such as low-molecular weight gelatine, will allow the DNA to diffuse more freely through to the light sensitive layer.

The nature of the irradiation procedure is also important. This procedure determines of how the light energy is absorbed by the chromophore and the energy is then transferred to the light sensitive materials. In competition to energy transfer through the chromophore direct absorption of light and heat energy by the light sensitive surfaces causes background fogging. Background versus chromophore depending energy deposition determines critically the signal to noise ratio. We use in our experiments preferably continuous irradiation, e.g. with a wolfram lamp for up to e.g. 1 min, which the best results obtained when we keep the irradiation time short, e.g. 10-45 sec. Alternatively we use a flash lamp, which exited for a very short time period, generally we can say short is better than longer irradiation times.

The reporter molecule may be any reporter molecules suitable for the detection of an analyte, e.g. nucleic acid molecules, nucleic acid analogue molecules or peptides. Especially preferred reporter molecules are described in co-assigned U.S. provisional application No. 60/855,574 “Click-Chemistry for the Production of Reporter Molecules” filed on 31 Oct. 2006, the content of which is herein incorporated by reference.

In a further preferred embodiment the detection involves irradiating a photosensitive medium in the presence of a sample suspected to contain the analyte and a reporter molecule, wherein the reporter molecule comprises photosensitizer groups and quencher groups capable of effecting an energy transfer to the photosensitive medium wherein marker groups may be formed in the medium. In the absence of analyte the photosensitizer group is quenched. In the presence of analyte, the quenching of the photosensitizer group is reduced or terminated. In this case, the photosensitizer group may induce the formation of marker groups, e.g. metal atoms or metal atom clusters in the photosensitive medium upon irradiation.

More preferably, the reporter molecule is a Molecular Beacon (MB) [12]. Molecular beacons are single-stranded hybridization probes, e.g. nucleic acid or nucleic acid analogue probes that form a stem-and-loop structure. The loop may contain a probe sequence that is complementary to a target sequence, and the stem is formed by the annealing of complementary arm sequences that are located on either side of the probe sequence. A photosensitizer, e.g. a fluorophore is covalently linked to the end of one arm and a quencher is covalently linked to the end of the other arm. Molecular beacons do not fluoresce when they are free in solution. However, when they hybridize to a nucleic acid strand containing a target sequence they undergo a conformational change that enables them to fluoresce brightly.

Many “fluorophores” and quenchers used for these probes are the same dyes used in black-and-white photography as spectral sensitizers [13], e.g. cyanine, merocyanine or pinacyanol dyes. The MB working principle can be summarized as follows: In the absence of targets, the probe is dark, because the stem places the fluorophore so close to the nonfluorescent quencher that they transiently share electrons, eliminating the ability of the fluorophore to fluoresce. When the probe encounters a target molecule, it forms a probe-target hybrid that is longer and more stable than the stem hybrid. The rigidity and length of the probe-target hybrid precludes the simultaneous existence of the stem hybrid. Consequently, the Molecular Beacon undergoes a spontaneous conformational reorganization that forces the stem hybrid to dissociate and the fluorophore and the quencher to move away from each other, restoring fluorescence.

The present invention verifies the correlation between the fluorescence measurements of a MB in its closed and open form and the relative signals detected on a modified photopaper. This technique is called Molecular Beacon based-DNA-Photography (MBDP).

The length of Molecular Beacon reporter molecules is preferably 15-100 nucleotides and more preferably 20-60 nucleotides. The Molecular Beacon molecules may be selected from nucleic acids such as DNA or RNA molecules or from nucleic acid analogues. The reporter molecules, e.g. the Molecular Beacon molecule may be manufactured according to standard procedures.

The sample may be any sample which may contain the analyte to be detected. For example, the sample may be a biological sample, such as an agricultural sample, e.g. a sample comprising plant material and/or material associated with the site where plants grow, plant materials are stored or processed. On the other hand, the sample may also be a clinical sample, such as a tissue sample or a body fluid sample such as blood, serum, plasma, etc, particularly of human origin. Further types of samples include, but are not limited to, environmental samples, soil samples, food samples, forensic samples or samples from valuable goods which are tested for brand protection.

Due to its high sensitivity, the method of the present invention is suitable for detecting analytes directly without amplification. According to the invention, even minute amounts of analytes, e.g. of nucleic acids, e.g. 0.1 ng or lower, preferably 0.01 ng or lower, more preferably 1 pg or lower, still more preferably 0.1 pg or lower, even more preferably 0.01 pg or lower and most preferably 0.001 pg or lower may be determined even without amplification.

The high sensitivity of the method of the present invention allows for the detection of analytes in the picomolar range and it is even possible to detect analytes in the zeptomolar range. An analysis in the zeptomolar range allows for the detection of single DNA molecules.

In a preferred embodiment of the invention, a sequence-specific detection of the analyte is carried out, wherein for example a nucleic acid having a specific sequence is distinguished from other nucleic acid sequences in the sample or a polypeptide capable of binding a specific nucleic acid sequence is distinguished from other polypeptides in the sample. Such a sequence-specific detection preferably comprises a sequence-specific hybridization reaction by which the nucleic acid sequence to be detected is associated with the reporter molecule.

The detection involves contacting the analyte and the reporter molecule comprising a photosensitizer group with a modified photosensitive medium as described above, e.g. by transferring a sample or sample aliquot in which an association product may be present onto the photosensitive medium, e.g. by spotting, pipetting etc.

Alternatively, the photosensitive medium may be modified by functionalization, e.g. by pre-impregnation with one or more reporter molecules. After contacting the sample with the functionalized photosensitive medium, an association product between reporter molecule and analyte may be formed in the photosensitive medium depending on the presence of analyte in the sample. In this embodiment of the invention, a photosensitive array comprising a plurality of different reporter molecules localized on individual predetermined sites of the photosensitive medium may be used to carry out a parallel detection of different analytes, e.g. different DNA analytes. The reporter molecules are preferably Molecular Beacons in this embodiment.

Upon irradiation, an energy transfer from the photosensitizer group to the photosensitive medium is effected such that marker groups, particularly latent images, such as metal, e.g. silver, nuclei are formed in the photosensitive medium in the presence, but not in the absence, of photosensitizer groups. If necessary, the marker groups, e.g. latent images, may be subjected to a development procedure, e.g. a chemical or photochemical development procedure according to photographic techniques. The photosensitive medium may be any solid support or any supported material capable of forming marker groups, e.g. metal nuclei. Preferably, the photosensitive medium is a light sensitive medium, such as light sensitive paper or a light sensitive emulsion or gel on a supportive material. More preferably the photosensitive medium is a photographic medium such as photographic paper. Irradiation is carried out under conditions, e.g. of wavelengths and/or intensity of irradiation light, under which selective marker group formation takes place in the presence of photosensitizer groups. Preferably, irradiation takes place with infrared light and/or with long wave visible light, depending on the sensitivity of the medium. The irradiation wavelength may be e.g. 500 nm or higher, 520 nm or higher, 540 nm or higher, 560 nm or higher, 580 nm or higher for visible light or 700 nm to 10 μm, for infrared light.

The photosensitizer group is a group which is capable of effecting an energy transfer, e.g. a transfer of light energy, to a photosensitive medium, i.e. a photographic medium such as photographic paper. The photosensitizer groups may be selected from known fluorescent and/or dye labelling groups such as cyanine-based indoline groups, quinoline groups, for example commercially available fluorescent groups such as Cy5 or Cy5.5.

The quencher group is a group capable of quenching the energy transfer from the photosensitizer group to the photosensitive medium. Preferably, the quencher group is capable of quenching the transfer of light energy. The quencher groups may be selected from known quencher groups, e.g. quencher groups known in Molecular Beacon reporter molecules, for example as described in references [12-16] which are herein incorporated by reference.

In certain embodiments, the reporter molecule may comprise a handle group, i.e. a group for introducing a photosensitizer group by reaction with a suitable reaction partner, i.e. a compound comprising one of the above groups. In a preferred embodiment, the handle groups are selected from Click functionalized groups, i.e. groups which may react with a suitable reaction partner in a cycloaddition reaction wherein a cyclic, e.g. heterocyclic linkage between the Click functional group and the reaction partner is formed, and wherein the reaction partner comprises a photosensitizer group. An especially preferred example of such a Click reaction is a (3+2) cycloaddition between azide and alkyne groups which results in the formation of 1,2,3-triazole rings. Thus, photosensitizer groups may be generated by performing a Click reaction of an azide or alkyne handle group and a corresponding reaction partner, i.e. a reaction partner comprising the complementary alkyne or azide group and additionally a photosensitizer group.

Preferably, the reporter molecule is a nucleic acid molecule, more preferably a single-stranded nucleic molecule. The term “nucleic acid” according to the present invention particularly relates to ribonucleotides, 2′-deoxyribonucleotides or 2′,3′-dideoxyribonucleotides. Nucleotide analogues may be selected from sugar- or backbone modified nucleotides, particularly of nucleotide analogs which can be enzymatically incorporated into nucleic acids. In preferred sugar-modified nucleotides the 2′-OH or H-group of the ribose sugar is replaced by a group selected from OR, R, halo, SH, SR, NH₂, NHR, NR₂ or CN, wherein R is C₁-C₆ alkyl, alkenyl or alkynyl and halo is F, Cl, Br or I. The ribose itself can be replaced by other carbocyclic or heterocyclic 5- or 6-membered groups such as a cyclopentane or a cyclohexene group. In preferred backbone modified nucleotides the phospho(tri)ester group may be replaced by a modified group, e.g. by a phosphorothioate group or a H-phosphonate group. Further preferred nucleotide analogues include building blocks for the synthesis of nucleic acid analogs such as morpholino nucleic acids, peptide nucleic acids or locked nucleic acids.

In a preferred embodiment, the methods and the reagent kits of the present invention are used for agricultural applications. For example, the invention is suitable for the detection of nucleic acids from plants, plant pathogens or plant pests such as viruses, bacteria, fungi or insects. Further, the invention is suitable for detecting genetic variabilities, e.g. SNPs in plants or plant parts, plant pathogens or plant pests such as insects.

A further application is a detection or monitoring of herbicide, fungicide or pesticide resistances, tolerances or intolerances, e.g. resistances, tolerances or intolerances in fungi, insects or plants in organisms or populations of organisms. The invention is also suitable for rapid genotyping, e.g. for the rapid detection and/or differentiation of species or strains of fungi, insects, or plants. Further, detection and/or differentiation of genetically modified organisms for strains, e.g. organisms or strains of fungi, insects or plants is possible.

The method of the invention is in particular suitable for the detection and characterisation of plants or seeds. In particular, by using the method of the invention or a test kit or test stripe adapted thereto, it is possible to analyse a product, e.g. a plant or a seed with regard to the manufacturer, with regard to the type of product and with regard to compounds or contents being contained in the product. It is particularly possible to detect from where and in particular, from which manufacturer an analyte comes from. This is possible because even minor differences or deviations from a wildtype, e.g. from a plant wildtype, may be detected by the method according to the present invention. Further, it is possible with the method according to the present invention to detect if and to what extent an analyte has been genetically engineered. It is further possible to detect if an analyte contains a certain resistance gene or if an analyte contains another characteristic due to genetic engineering. Such modifications often comprise only the replacement of one or two bases. But even such minor modifications may be detected with the method according to the present invention. The method according to the invention makes it possible to define the product itself, i.e. to find out whether it is wheat, rapeseed, rice etc. It is finally possible to define the resource content or rather the content of certain agents. It is, for example, possible to determine the oil content in rapeseed or the presence of a gene that is resistant to drought stress. The method according to the invention may therefore be used for the control and monitoring of the characteristics of a product, especially of promised characteristics of a product. Such an application is especially useful in the field of nutrients but also in pharmaceuticals. It is possible with the method according to the present invention to assess plants that are produced and distributed by plant farming with regard to their origin and their actual characteristics.

Especially preferred is a test kit or a test strip, which allows for the control and allocation of products or product characteristics.

Due to the high sensitivity of the invention, early diagnostic of pathogens is possible, i.e. diagnostics before first symptoms of the presence of pathogens is visible. This is particularly important for the diagnosis of soy rust (Phakospora pachyrizi) or other pathogens, e.g. Blumeria graminis, Septoria tritici or Oomycetes or other pathogens for which control is only possible, if their presence is detected before it can be visually recognized.

Further, the invention is suitable for medical, diagnostic and forensic applications, e.g. in human or veterinary medicine, e.g. for the detection of nucleic acids from pathogens, e.g. human pathogens or pathogens of livestock or pet animals. In particular, it is possible to detect e.g. viruses or bacteria.

Further preferred applications include the detection of genetic variabilities, e.g. SNPs in humans or the detection of medicament resistances, tolerances or intolerances or allergies. Further, the invention is suitable for genotyping, particularly genotyping of humans in order to determine mutations associated with predisposition or enhanced risk of disorders, allergies and intolerances. The invention may also be used for the detection of genetically modified organisms or strains, organisms or strains of bacteria or viruses but also genetically modified life stock animals etc. The invention is particularly suitable for the rapid diagnosis of diseases, e.g. genetic diseases, allergic diseases, autoimmune diseases or infectious diseases.

Furthermore, the invention is suitable for detecting the function and/or expression of genes, e.g. for research purposes.

Still a further embodiment is the use of the method for brand protection, e.g. for detecting specific information encoded in products such as valuable goods like plant protection products, pharmaceuticals, cosmetics and fine chemicals (e.g. vitamins and amino acids) and beverage products, fuel products, e.g. gasoline and diesel, consumer electronic appliances can be marked. Further, packaging of these and other products can be marked. The information is encoded by nucleic acids or nucleic acid analogues which have been incorporated into the product and/or into the packaging of a product. The information may relate to the identity of the manufacturer, to production sites, date of production and/or distributor. By means of the present invention, rapid detection of product-specific data can be carried out. A sample may be prepared from an aliquot of the product which is then contacted with one or several sequence-specific functionalized hybridization probes capable of detecting the presence of nucleic acid-encoded information in the sample.

The invention is also suitable for the field of nutrients. For example, in the feed area, animal nutrients, e.g. corn, are supplemented with a greater quantity of preservatives such as propionic acid. By applying the method of the invention, the addition of preservatives can be reduced. Further, genomic analysis with the method of the invention allows the prediction of an individual's capability to utilize specific nutrients (nutrigenomics).

### FIGURE LEGENDS

FIG. 1: Working principle of Molecular Beacons. a) two different pathways to denature the hairpin structure of MBs, by a target annealed to the loop region of the hairpin (top) and by temperature, denaturing reagent or ssDNA binding proteins (bottom). b) a typical fluorescence/temperature spectrum of a MB in its open (upper line) and closed (lower line) form.

FIG. 2: Schematic representation of the DNA-photography working principle based on MBs, MBDP. Only the mixture to analyze in which MB is annealed with the target T gives a positive signal as a black spot in the photopaper. The closed form of MB gives no signal on the photopaper.

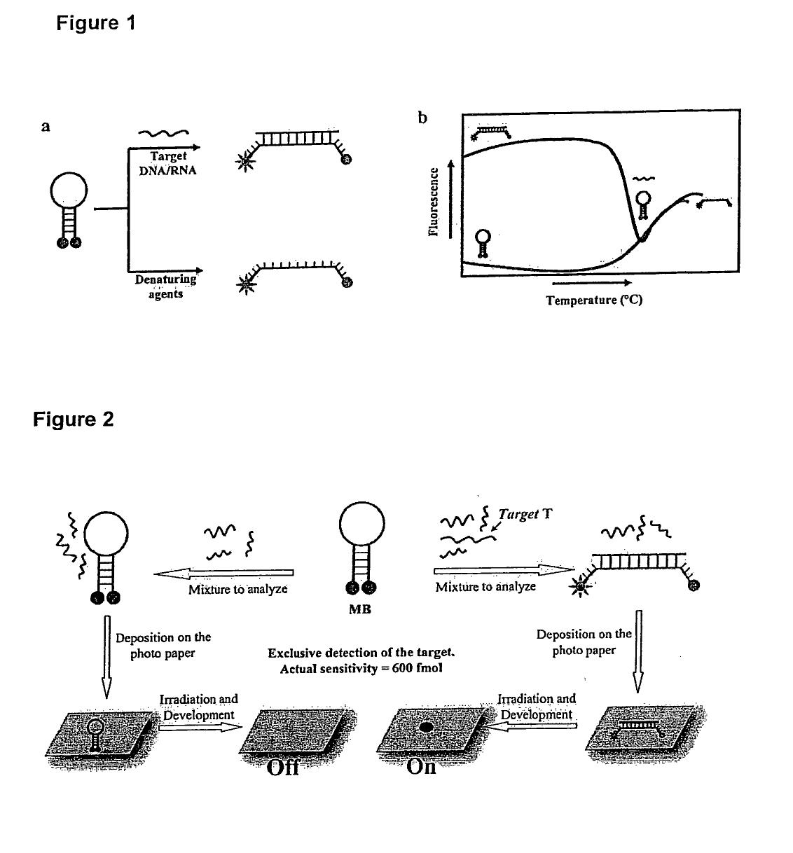

### CONCLUSIONS

The present invention describes a novel method to detect biomolecules using the principle of black-and-white photography. Picomolar sensitivity levels can be achieved without extensive optimization. The technique is based on the highly specific hybridization properties of DNA. Preliminary experiments show that this technique is easy to use and inexpensive although astonishing results could already be achieved with it. So far our detection limit using the above mentioned commercial photopaper and the conditions reported here is 600 femtomoles of target T per 1 μL of solution analyzed. This limit is dependent on the nature of the salts and the photopaper used, and can be modulated by using different dyes and different light sources. The detection of a selected DNA-sequence in the nanomolar range (femtomoles of target) is an astonishing result for such an easy and quick method.

Different targets can be detected using different MBs at the same time since the specificity of these probes is well established in literature [16]. MBs are indeed applied in single nucleotide polymorphism (SNP) studies and in multiplex detection of different targets as well [17]. Reported modification of the MBs structure as in the locked nucleic acid based MBs (LNA-MBs) [18] or of the dye/quencher couples as for MBs with superquenchers [19] or with gold-quenchers [17] make these MBs the perfect candidates for many applications. Additionally it would even be possible to design specific photopaper stripes in which many MBs are already absorbed. By using different light sources (or different filters) for each MB it would be possible to detect different specific targets simultaneously. Moreover, multiply-modified MBs could be designed and synthesized using the click-chemistry functionalization of DNA developed in our labs [20], thus drastically increasing the availability of specific MBs in a modular and practical fashion.

The content of the documents cited in the present application is herein incorporated by reference.

### REFERENCES

1. R. K. Saiki, S. S., F. Faloona, K. B. Mullis, G. T. Horn, H. A. Erlich, N. Arnheim Enzymatic amplification of beta-globin genomic sequences and restriction site analysis for diagnosis of sickle cell anemia. Science 1985, 230, 1350-1354.
2. Taton, T. A.; Mirkin, C. A.; Letsinger, R. L. Scanometric DNA Array Detection with Nanoparticle Probes. Science 2000, 289, 1757-1760.
3. Park, S.-J.; Taton, T. A.; Mirkin, C. A. Array-Based Electrical Detection of DNA with Nanoparticle Probes. Science 2002, 295, 1503-1506.
4. Fujishima, T.; Zhaopeng, L.; Konno, K.; Nakagawa, K.; Okano, T.; Yamaguchi, K.; Takayama, H. Highly Potent Cell Differentiation-Inducing Analogues of 1,25-Dihydroxyvitamin D3: Synthesis and Biological Activity of 2-Methyl-1,25-dihydroxyvitamin D3 with Side-Chain Modifications. Bioorg. Med. Chem. 2001, 9, 525-535.
5. Nam, J. M.; Stoeva, S. I.; Mirkin, C. A. Bio-Bar-Code-Based DNA Detection with PCR-like Sensitivity. J. Am. Chem. Soc. 2004, 126, 5932-5933.
6. Rosi, N. L.; Mirkin, C. A. Nanostructures in Biodiagnostics. Chem. Rev. 2005, 105, 1547-1562.
7. Baker, E. S.; Hong, J. W.; Gaylord, B. S.; Bazan, G. C.; Bowers, M. T. PNA/dsDNA Complexes: Site Specific Binding and dsDNA Biosensor Applications. J. Am. Chem. Soc. 2006, 128, 8484-8492.
8. Lewis, F. D.; Wu, T.; Zhang, T.; Letsinger, R. L.; Greenfield, S. R.; Wasielewski, M. R. Distance-Dependent Electron Transfer in DNA Hairpins. Science 1997, 277, 673-676.
9. Ciuffreda, P.; Casati, S.; Santaniello, E. The Action of Adenosine Deaminase (E.C. 3.5.4.4.) on Adenosine and Deoxyadenosine Acetates: The Crucial Role of the 5′-Hydroxy Group for the Enzyme Activity. Tetrahedron 2000, 56, 3239-3243.
10. Vogel, H. M. Berichte 1873, 6, 1302.
11. West, W.; Gilman, P. B. The Theory of the Photographic Process; T. H. James ed.; Macmillan: New York, 1977.
12. Tyagi, S.; Kramer, F. R. Molecular Beacons: Probes that Fluoresce upon Hybridization. Nature Biotechnology 1996, 14, 303-308.
13. Marras, S. A. E.; Kramer, F. R.; Tyagi, S. Efficiencies of fluorescence resonance energy transfer and contact-mediated quenching in oligonucleotide probes. Nucleic Acids Research 2002, 30, e122.
14. Varma-Basil, M.; El-Hajj, H.; Marras, S. A. E.; Hazbon, M. H.; Mann, J. M.; Connell, N. D.; Kramer, F. R.; Alland, D. Molecular Beacons for Multiplex Detection of Four Bacterial Bioterrorism Agents. Clin Chem 2004, 50, 1060-1062.
15. Tan, W.; Wang, K.; Drake, T. J. Molecular beacons. Current Opinion in Chemical Biology 2004, 8, 547-553.
16. Marras, S. A. E.; Tyagi, S.; Kramer, F. R. Real-time assays with molecular beacons and other fluorescent nucleic acid hybridization probes. Clinica Chimica Acta 2006, 363, 48-60.
17. Dubertret, B.; Calame, M.; Libchaber, A. J. Single-mismatch detection using gold-quenched fluorescent oligonucleotides. Nat Biotech 2001, 19, 365-370.
18. Wang, L.; Yang, C. J.; Medley, C. D.; Benner, S. A.; Tan, W. Locked Nucleic Acid Molecular Beacons. J. Am. Chem. Soc. 2005, 127, 15664-15665.
19. Yang, C. J.; Lin, H.; Tan, W. Molecular Assembly of Superquenchers in Signaling Molecular Interactions. J. Am. Chem. Soc. 2005, 127, 12772-12773.
20. Gierlich, J.; Burley, G. A.; Gramlich, P. M. E.; Hammond, D. M.; Carell, T. Click Chemistry as a Reliable Method for the High-Density Postsynthetic Functionalisation of Alkyne-Modified DNA. Org. Lett. 2006, 8, 3639-3642.

1-23. (canceled)

24. A method for detecting an analyte in a sample comprising the steps:
(i) providing a sample,
(ii) providing a reporter molecule comprising a photosensitizer group or a handle group for introducing a photosensitizer group,
(iii) contacting the sample with the reporter molecule,
(iv) irradiating said reporter molecule in contact with a modified photosensitive medium under conditions wherein marker groups are formed depending on the binding of the reporter molecule to the analyte and
(v) detecting said marker groups,
(vi) wherein when the providing step (ii) comprises a handle group, reacting the handle group with a reaction partner comprising a photosensitizer group.

25. The method of claim 24 wherein the analyte is selected from nucleic acids and nucleoside-, nucleotide- or nucleic acid-binding molecules.

26. The method of claim 24, wherein the analyte to be detected is a nucleic acid selected from DNA and RNA.

27. The method of claim 24, wherein the sample is a biological sample.

28. The method of claim 27, wherein the sample is an agricultural sample, nutritional sample or a clinical sample.

29. The method of claim 24, wherein the detection is carried out directly without amplification.

30. The method of claim 24, wherein the detection is carried out in combination with an amplification step.

31. The method of claim 24, wherein the reporter molecule is a nucleic acid molecule.

32. The method of claim 24, wherein the handle group is selected from the group consisting of azide and alkyne groups.

33. The method of claim 32, wherein said azide groups are reacted by performing a Click reaction with an alkyne group of a reaction partner comprising a photosensitizer group.

34. The method of claim 32, wherein said alkyne groups are reacted by performing a Click reaction with an azide group of a reaction partner comprising a photosensitizer group.

35. The method of claim 24, wherein the photosensitizer groups are selected from the group consisting of fluorescent dye groups.

36. The method of claim 35, wherein the photosensitizer groups are selected from the group consisting of cyanine-based indoline groups and quinoline groups.

37. The method of claim 24, wherein the photosensitive medium comprises metal atoms or ions capable of forming metal nuclei.

38. The method of claim 37, wherein the metal is Ag.

39. The method of claim 24, wherein the photosensitive medium is a light sensitive paper comprising a photographic paper, a light sensitive emulsion, a gel on a supportive material, or any combination thereof.

40. The method of claim 24, wherein the irradiating step (v) is carried out with long wave visible light and/or with infrared light.

41. A reagent kit for detecting an analyte in a sample comprising:
(a) a reporter molecule comprising a photosensitizer group or a handle group for introducing a photosensitizer group, and
(b) a modified photosensitive medium which forms marker groups upon irradiation of photosensitizer groups,
(c) wherein when the reporter molecule comprises a handle group, the kit further comprises a reaction partner for the handle group comprising a photosensitizer group.

42. The kit of claim 41, wherein the reporter molecule is present as reagent impregnated on the photosensitive medium.

43. The method of claim 24, wherein the method is used for agricultural applications, for medical, diagnostic, or forensic applications, for detecting function and/or expression of a gene, for brand protection or for nutritional applications.

44. The method of claim 43, wherein nutritional application is in the feed area.

45. The method of claim 24 for detecting an analyte which has been modified by genetic engineering.

46. The method claim 24 for detecting an analyte which is a product of a genetically modified organism.
